(19) United States
(12) Patent Application Publication
(10) Pub. No.: US 2024/0008862 A1
(43) Pub. Date: Jan. 11, 2024

(54) RETRACTOR SYSTEM AND RETRACTOR ARM WITH DETACHABLE HANDLE

(71) Applicant: Thompson Surgical Instruments, Inc., Traverse City, MI (US)

(72) Inventor: Daniel K. Farley, Traverse City, MI (US)

(21) Appl. No.: 18/370,662

(22) Filed: Sep. 20, 2023

Related U.S. Application Data
(63) Continuation-in-part of application No. 17/163,882, filed on Feb. 1, 2021, now Pat. No. 11,806,002.

(51) Int. Cl. A61B 17/02; A61B 90/50

(52) U.S. Cl. CPC A61B 17/02; A61B 90/50; A61B 2017/00477

(57) ABSTRACT

A retractor system may include a mounting assembly, an articulated arm, and a retractor blade. The mounting assembly may include a post and a bed mount. The articulated arm may include arm segments, a retractor connector, and a handle segment. The retractor blade may be attached to the retractor connector. A proximal end of the arm segments may be coupled to the post. A proximal end of the handle segment may be coupled to a distal end of the arm segments. A distal end of the handle segment may be coupled to the retractor connector. The handle segment may include a handle that extends longitudinally along the handle segment.

### RELATED APPLICATIONS

The present application is a continuation-in-part of U.S. patent application Ser. No. 17/163,882, filed Feb. 1, 2021, the disclosure of which is incorporated herein by reference in its entirety.

### BACKGROUND

The present disclosure relates to a surgical apparatus that retracts soft tissue and other anatomy of a patient in order to provide access to an operative site.

During a surgical procedure, a surgeon may make an incision in a patient to access internal organs, bones, and/or other anatomical structures. Retraction devices may be used to hold back soft tissue and other patient anatomy in the immediate area of the incision. Such retraction devices may provide the surgeon with an unobstructed view of the internal organs, bones, and/or other anatomical structures. Furthermore, the retraction devices may provide the surgeon with an opening via which the surgeon may access the anatomical structures with one or more surgical tools.

Further limitations and disadvantages of conventional and traditional approaches will become apparent to one of skill in the art, through comparison of such approaches with the present disclosure as set forth in the remainder of the present application with reference to the drawings.

### SUMMARY

Various aspects of this disclosure provide a retractor system comprising retractor blades that retract anatomy to provide exposure of an operative site. For example and without limitation, various aspects of this disclosure are directed to a retractor system with one or more articulating arms to which retractor blades are attached. The articulating arms may position retractor blades in a surgical site and retain the retractor blades in a desired position. Detachable handles may be attached to the articulating arms to aid in positioning the articulating arms and/or retractor blades attached therefore. After positioning the articulating arms and/or retractor blades, the handle may be detached and removed in order to provide less encumbered access to the surgical site. In some embodiments, the articulated arm may include a handle segment with an integral handle instead of a detachable handle.

### BRIEF DESCRIPTION OF THE DRAWINGS

FIG. 1 provides a perspective view of a retractor system in accordance with various aspects of the present disclosure.

FIGS. 2A and 2B provide perspective views of the retractor system of FIG. 1.

FIG. 3A provides a cross section view of the retractor connector, handle arm segment, and handle attached to a handle arm segment of FIG. 1.

FIG. 3B provides a cross section view of the retractor connector, handle arm segment, and the handle detached from the handle arm segment of FIG. 1.

FIGS. 4A-4C depicted various details of the handle of the retractor system of FIG. 1.

FIG. 5 provides a perspective view of a retractor blade and its attachment post suitable for use with the retractor system of FIG. 1.

FIG. 6 provides a detailed perspective view of the handle mount of the handle arm segment.

FIG. 7 provides a perspective view of another retractor system in accordance with various aspects of the present disclosure.

FIGS. 8A and 8B provide a top view and corresponding cross section view of the retractor system of FIG. 7 with a detached retractor blade.

FIGS. 9A and 9B provide a top view and corresponding cross section view of the retractor system of FIG. 7 with an attached retractor blade in a rotated position.

FIG. 10 provide a cross section view of the handle segment of the retractor system of FIG. 7 with a retractor blade detached from its retractor connector.

FIG. 11 provides a perspective view of another retractor system in accordance with various aspects of the present disclosure.

FIGS. 12A and 12B provide a top view and corresponding cross section view of the retractor system of FIG. 11 with a detached retractor blade.

FIGS. 13A and 13B provide a top view and corresponding cross section view of the retractor system of FIG. 11 with an attached retractor blade in a rotated position.

FIG. 14 provide a cross section view of the handle segment of the retractor system of FIG. 11 with a retractor blade detached from its retractor connector.

FIG. 15 provides a perspective view of another retractor system in accordance with various aspects of the present disclosure.

FIGS. 16A and 16B provide a top view and corresponding cross section view of the retractor system of FIG. 15 with a detached retractor blade.

FIGS. 17A and 17B provide a top view and corresponding cross section view of the retractor system of FIG. 15 with an attached retractor blade in a rotated position.

FIG. 18 provide a cross section view of the handle segment of the retractor system of FIG. 15 with a retractor blade detached from its retractor connector.

### DETAILED DESCRIPTION

The following discussion presents various aspects of the present disclosure by providing examples thereof. Such examples are non-limiting, and thus the scope of various aspects of the present disclosure should not necessarily be limited by any particular characteristics of the provided examples. In the following discussion, the phrases “for example,” “e.g.,” and “exemplary” are non-limiting and are generally synonymous with “by way of example and not limitation,” “for example and not limitation,” and the like.

As utilized herein, “and/or” means any one or more of the items in the list joined by “and/or”. As an example, “x and/or y” means any element of the three-element set {(x), (y), (x, y)}. In other words, “x and/or y” means “one or both of x and y.” As another example, “x, y, and/or z” means any element of the seven-element set {(x), (y), (z), (x, y), (x, z), (y, z), (x, y, z)}. In other words, “x, y and/or z” means “one or more of x, y, and z.”

The terminology used herein is for the purpose of describing particular examples only and is not intended to be limiting of the disclosure. As used herein, the singular forms are intended to include the plural forms as well, unless the context clearly indicates otherwise. It will be further understood that the terms “comprises,” “includes,” “comprising,” “including,” “has,” “have,” “having,” and the like when used in this specification, specify the presence of stated features, integers, steps, operations, elements, and/or components, but do not preclude the presence or addition of one or more other features, integers, steps, operations, elements, components, and/or groups thereof.

It will be understood that, although the terms first, second, etc. may be used herein to describe various elements, these elements should not be limited by these terms. These terms are only used to distinguish one element from another element. Thus, for example, a first element, a first component, or a first section discussed below could be termed a second element, a second component, or a second section without departing from the teachings of the present disclosure. Similarly, various spatial terms, such as “upper,” “lower,” “side,” and the like, may be used in distinguishing one element from another element in a relative manner. It should be understood, however, that components may be oriented in different manners, for example a device may be turned sideways so that its “top” side is facing horizontally and its “side” side is facing vertically, without departing from the teachings of the present disclosure. Additionally, the term “on” will be utilized in the document to mean both “on” and “directly on” (e.g., with no intervening component).

In the drawings, various dimensions (e.g., thicknesses, widths, etc.) may be exaggerated for illustrative clarity. Additionally, like reference numbers are utilized to refer to like elements through the discussions of various examples.

The discussion will now refer to various example illustrations provided to enhance the understanding of the various aspects of the present disclosure. The scope of this disclosure is not limited by specific characteristics of the examples provided and discussed herein.

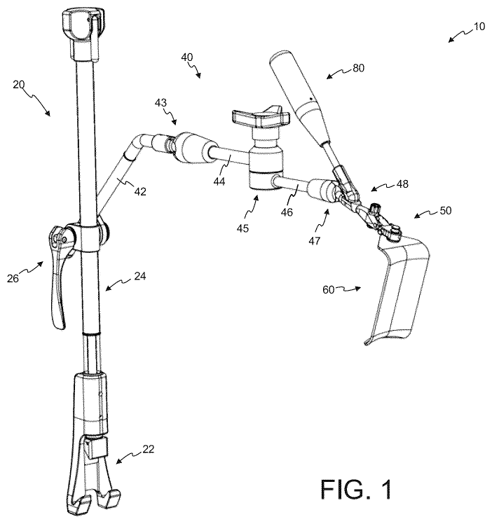

FIG. 1 illustrates an embodiment of a retractor system 10 in accordance with various aspects of the present disclosure. The retractor system 10 may include a mounting assembly 20, an articulated arm 40, a retractor blade 60, and a handle 80. In particular, FIG. 1 depicts a single mounting assembly 20 with a single articulated arm 40 attached to the mounting assembly 20. However, in some embodiments, the retractor system 10 may include a different number of mounting assemblies 20 and/or a different number of articulated arms 40 attached to each mounting assembly 20.

As shown, the retractor system 10 may include a retractor blade 60 and a handle 80 attached to each articulated arm 40. However, in some embodiments, the retractor system 10 may include a different number of handles 80 and/or a different number of retractor blades 60. For example, a person may use the handle 80 to position one articulated arm 40 and then detach the handle 80 from the positioned articulated arm 40. The person may then attach the handle 80 to another articulated arm 40 and use the newly attached handle 80 to position the another articulated arm 40.

Each mounting assembly 20 may include a bed mount 22, a post 24, and a clamp 26. In particular, the bed mount 22 of each mounting assembly 20 may affix a respective post 24 to a hospital bed (not shown). In particular, the post 24 may be affixed such that the post 24 extends upward from the bed mount 22 in a generally vertical direction. Each post 24 may provide a location to which a person may secure an articulated arm 40. In particular, the articulated arm 40 may be affixed to the post 24 via the clamp 26. When its handle is in a released position, the clamp 26 may slide along the length of the post 24 and adjust a height of the articulated arm 40. Moreover, the clamp 26 may permit a length of the articulated arm 40 to slide through the clamp 26 and effectively adjust a distance that a distal end of the articulated arm 40 is from the post 24. After appropriately adjusting the height and length of the articulated arm 40, the handle of clamp 26 may be moved to a locked position. In the locked position, the clamp may secure the articulated arm 40 to the post 24, prevent further sliding of the clamp 26 along the post 24, and prevent further sliding of the articulated arm 40 through the clamp 26.

Each articulated arm 40 is adapted to adjustably constrain a retractor blade 60 secured to the distal end of the articulated arm 40. In some embodiments, the articulated arm 40 may provide the retractor blade 60 with at least some freedom of movement relative to the post 24 and the bed mount 22. In particular, the articulated arm 40 may allow the retractor blade 60 some limited movement but may generally maintain the retractor blade 60 at or near a selected position. In other embodiments, the articulated arm 40 may be adapted to maintain the retractor blade 60 in a substantially fixed or stationary position once the articulated arm 40 is positioned and locked into place.

To this end, the articulated arm 40 may comprise multiple arm segments and joints that permit articulating the arm segments. In particular, the articulated arm 40 may include a shoulder arm segment 42. A distal end of the shoulder arm segment 42 may be joined to a proximal end of an upper arm segment 44 by a shoulder joint 43. Moreover, a distal end of the upper arm segment 44 may be joined to a proximal end of a fore arm segment 46 by an elbow joint 45. Finally, a distal end of the fore arm segment 46 may be joined to a proximal end of a handle arm segment 48 by a wrist joint 47.

The arm segments 42, 44, and 46 are generally referred to herein as a “shoulder” arm segment, an “upper” arm segment, a “fore” arm segment in a manner similar to a human arm. Similarly, joints 43, 45, 47 are generally referred to herein as a “shoulder” joint, an “elbow” joint, and a “wrist” joint in a manner similar to joints of a human arm. However, such terms are merely used as a manner of convenience. This analogy to a human arm is especially helpful with regard to the depicted embodiment in which the joints 43, 47 are ball joints and the joint 45 is a pivot joint. Such joints provide the articulated arm 40 with articulation similar to the human arm. However, the articulated arm 40, in some embodiments, may include a fewer number of arm segments or a greater number of arm segments. Similarly, the joints of the articulated arm 40 may not align with joints of the human arm and may comprise some combination of single-axis hinge joints, pivot joints, universal joints, ball joints, prismatic joints, and/or other types of joints.

The arm segments 44, 46, 48 may each comprise one or more generally straight and generally cylindrical members. Similarly, the shoulder arm segment 42 may include one or more generally cylindrical members. However, unlike arm segments 44, 46, 48, the cylindrical members of the shoulder arm segment 42 may include one or more bends or curves. In some embodiments, each arm segment 42, 44, 46, 48 may comprise various straight and/or bent members so as to accommodate different procedures or access sites.

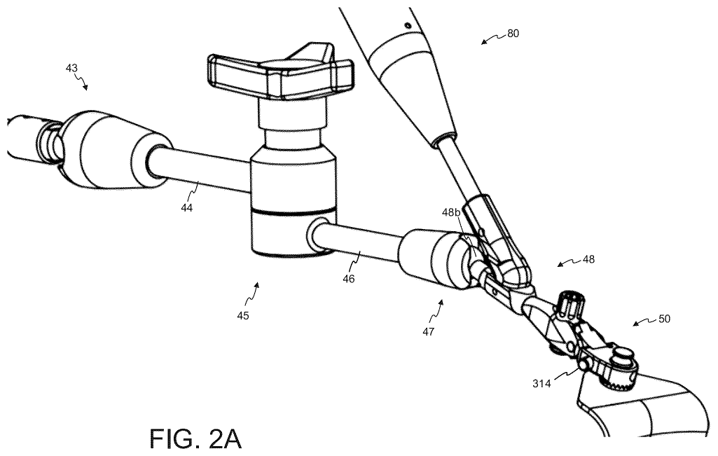

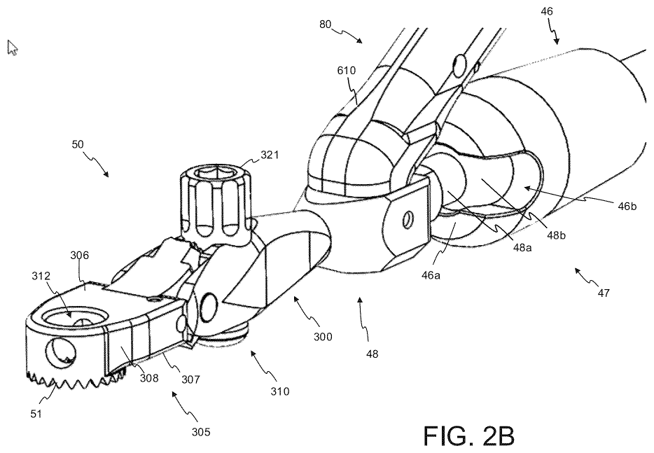

As shown in FIGS. 2A and 2B, the fore arm segment 46 and handle arm segment 48 are joined by the wrist joint 47. In particular, a distal end of the fore arm segment 46 may include a socket 46a of the wrist joint 47. Moreover, a proximal end of the handle arm segment 48 may include a shaft 48a, which is coupled to a ball 48b of the wrist joint 47. However, in other embodiments, the proximal end of the handle arm segment 48 may include the socket of the wrist joint 47 and the distal end of the fore arm segment 46 may include a shaft attached to a ball of the wrist joint 47.

In general, the ball 48b may engage internal sides of the socket 46a and the socket 46a may prevent the ball 48b from being withdrawn from socket 46a. However, the socket 46a may otherwise permit the ball 48b to rotate within the socket 46a and along the internal sides of the socket 46a. As such, the wrist joint 47 may permit articulation of the handle arm segment 48 with respect to the fore arm segment 46 about multiple axes or an indefinite number axes. In some embodiments, the wrist joint 47 may constrain or otherwise limit a degree of movement of the handle arm segment 48 about one or more axes. For example, as shown in FIG. 2B, the wrist joint 47 may be oriented such that an opening 46b of the socket 46a is narrower in the vertical direction than in the horizontal direction. In such an embodiment, upper and lower sides of the socket opening 46b may engage the shaft 48a and limit articulation of the handle arm segment 48 in an upward or downward direction. Conversely, left and right sides of the socket opening 46b may engage the shaft 48a and limit articulation of the handle arm segment 48 in a leftward or rightward direction, but permit greater articulation in such directions than what is permitted in the upward or downward directions.

FIG. 2A depicts an orientation of the wrist joint 47 in which the socket opening 46b permits greater articulation in the vertical direction than in the horizontal direction. FIG. 2A depicts that the shoulder joint 43 may be implemented in a manner similar to the wrist joint 47. In particular, FIG. 2A depicts an orientation of the shoulder joint 43 in which the socket opening of the shoulder joint 43 permits greater articulation in the horizontal direction than in the vertical direction.

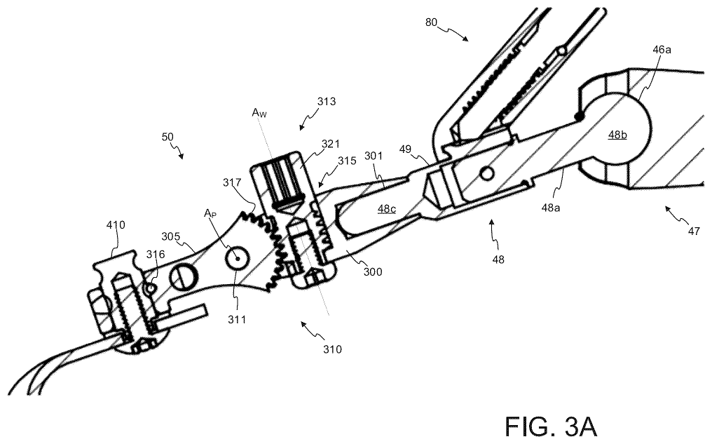

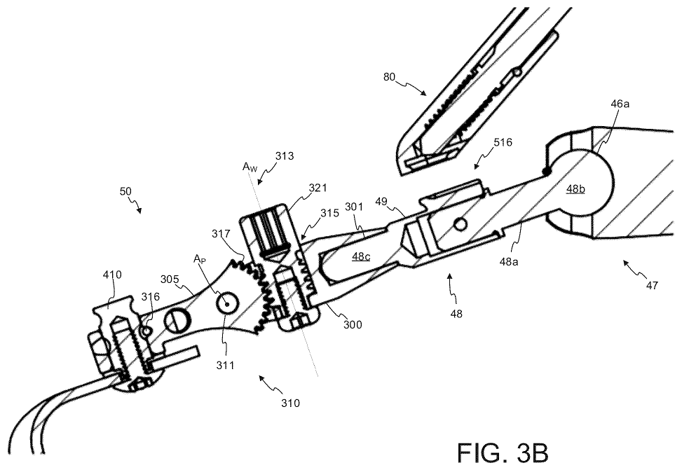

As shown, in FIGS. 3A and 3B, the retractor connector 50 may be at a distal end of the handle arm segment 48. The retractor connector 50 may comprise a connector body 300, a connector head 305, and an adjustable pivot 310. In particular, as shown in FIG. 3A, a cylindrical post 48c at the distal end of the handle arm segment 48 may be received by and affixed to a cylindrical port 301 at a proximal end of a connector body 300. Furthermore, a distal end of the connector body 300 may be coupled to a proximal end of the connector head 305 via the adjustable pivot 310.

The adjustable pivot 310 may comprise a pin 311 that passes through aligned barrel holes of connector body 300 and the connector head 305. The adjustable pivot 310 may comprise a worm drive 313. The worm drive 313 may control and adjust the amount of pivot or angle between the connector body 300 and the connector head 305. In particular, a thumb screw or worm 315 of the worm drive 313 may pass through a distal end of the connector body 300 such that a longitudinal axis Aw of the worm 315 is perpendicular to the longitudinal axis AP of the pin 311. Furthermore, a worm gear 317 of the worm drive 313 may be positioned along a proximal end of the connector head 305 such that teeth of the worm gear 317 mesh with threads of the thumb screw or worm 315. Rotation of the worm 315 via the worm head 321 in a first direction may adjust or force the connector head 305 in a downward direction with respect to the connector body 300. Conversely, rotation of the worm 315 in a second direction opposite the first direction may adjust or force the connector head 305 in an upward direction with respect to the connector body 300.

The connector head 305 may include an upper side 306, a lower side 307, and sidewalls 308 that couple the upper side 306 to the lower side 307. The connector head 305 may include an attachment port 312 through the upper side 306 and the lower side 307, a release button 314, and a locking ball 316. In the illustrated embodiment, the attachment port 312 (FIG. 2B), release button 314 (FIG. 2A), and locking ball 316 (FIG. 3A) cooperate to detachably couple and lock a retractor blade 60 to the articulated arm 40. Through the use of an internal mechanism (not shown), depressing the release button 314 allows increased movement of the locking ball 316, which permits an attachment post 410 of retractor blade 60 to be inserted into or removed from the attachment port 312. Releasing the release button 314 causes the release button 314 and internal mechanism to return to a locked position in which the internal mechanism forces the locking ball 316 into the attachment port 312 such that the locking ball 316 engages the attachment post 410 of the retractor blade 60 and secures the retractor blade 60 to the retractor connector 50. Thus, a retractor blade 60 may be secured to the articulated arm 40 and a hospital bed via a retractor connector 50.

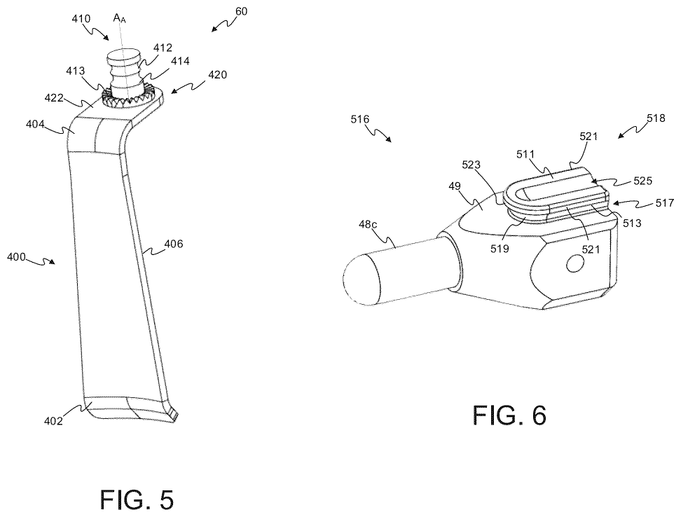

Referring now to FIG. 5, the illustrated retractor blade 60 includes a blade 400 having a distal end 402, a proximal end 404, and a retracting portion 406. The retractor blade 60 may include a base portion 420 having an attachment post 410. The distal end 402 may correspond to the end of the blade 400 oriented more deeply inside the patient during a surgical procedure, and the proximal end 404 may correspond to the end of the blade 400 oriented closer to a practitioner during a surgical procedure.

The retracting portion 406 generally extends from the proximal end 404 adjoined to the base portion 420 to the distal end 402. The retracting portion 406 may extend at an angle (e.g., 90°) from the base portion 420. The retracting portion 406 may be sized and adapted to hold back tissue from a site of interest during a procedure. In certain embodiments, the retractor system 10 may include a number of differently sized and/or shaped retractor blades 60 to provide increased adaptability for different procedures and/or patients.

The base portion 420, located proximate to the proximal end 404 of blade 400, may provide a location or locations to grasp and/or secure the retractor blade 60. Located proximate to the base portion 420 is the attachment post 410. The attachment post 410 may be sized and adapted to provide a location for attachment to connector head 305 of the retractor connector 50. In particular, the attachment post 410 may be sized and adapted to cooperate with the attachment port 312 of the retractor connector 50. To this end, the attachment post 410 may have a generally cylindrical-shape and may extend from an upper side 422 of the base portion 420. In one embodiment, a longitudinal axis A_{A} of the attachment post 410 extends at a right angle from the upper side 422 of the retractor blade 60. However, the attachment post 410 in some embodiments may extend from the upper side 422 at other angles.

As shown in FIG. 5, the upper side 422 of the base portion 420 may include serrations or teeth 413. Similarly, as shown in FIG. 2B, a lower side of the retractor connector 50 may include serrations or teeth 51. The teeth 413 of the retractor blade 60 may engage the teeth 51 of the connector head 305 of the retractor connector 50 when the lower annular groove 414 accepts the locking ball 316. The engaged teeth 51, 413 may restrict rotation of the attachment post 410 relative to the connector head 305.

As shown in FIG. 5, the attachment post 410 may include an upper annular groove 412 and a lower annular groove 414. The attachment post 410 and annular grooves 412, 414 may be sized such that the attachment post 410 may pass freely through the attachment port 312 of the retractor connector 50 when the locking ball 316 of the retractor connector 50 is not placed into a locked position by the internal mechanism. However, when the release button 314 permits the internal mechanism to return to a locked position, the locking ball 316 is forced into the attachment port 312 and engages one of annular grooves 412, 414.

In particular, the upper annular groove 412 may be sized and adapted such that when the upper annular groove 412 is engaged with the locking ball 316 in the locked position, the attachment post 410 is prevented from moving longitudinally through the attachment port 312. However, when locking ball 316 engages the upper annular groove 412, the upper annular groove 412 may position the upper side 422 away from a lower side of the retractor connector 50. thus disengaging the teeth 51 of the retractor connector 50 from the teeth 413 of the retractor blade 60. Thus, with the locking ball 316 engaged with the upper annular groove 412, the attachment post 410 and attached blade 400 may rotate about the longitudinal axis A_{A} with respect to the retractor connector 50.

Similar to the upper annular groove 412, the lower annular groove 414 may be sized and adapted such that when the lower annular groove 414 is engaged with the locking ball 316 in the locked position, the attachment post 410 is prevented from moving longitudinally through the attachment port 312. However, when locking ball 316 engages the lower annular groove 414, the lower annular groove 414 may position the upper side 422 of the retractor blade 60 such that the teeth 413 of the retractor connector 50 engage the teeth 413 of the retractor blade 60. Thus, with the locking ball 316 engaged with the lower annular groove 412, the engaged teeth 51, 413 may prevent the attachment post 410 and attached blade 400 from rotating about the longitudinal axis A_{A} with respect to the retractor connector 50. Thus, by positioning the attachment post 410 such that the locking ball 316 engages either the upper annular groove 412 or the lower annular groove 414, a person may alter the amount of freedom of movement of the retractor blade 60 relative to the retractor connector 50.

In the depicted embodiments, the attachment port 312 of the retractor connector 50 and the attachment post 410 of the retractor blade 60 are cylindrical with circular cross-sections. Such an embodiment may permit swiveling of the retractor blade 60 with regard to the retractor connector 50, even after attachment. However, such swiveling of the retractor blade 60 may not be required or may not be desired for certain surgical procedures. As such, the attachment port 312 and the attachment post 410 may be shaped in a manner that prevents such swiveling. For example, the attachment port 312 and the attachment post 410 may remain cylindrical but have a non-circular cross-section (e.g., square, rectangular, hexagonal, etc.). In yet other embodiments, the attachment port 312 and attachment post 410 may not by cylindrical, but may otherwise provide a mating engagement of the retractor blade 60 to the retractor connector 50. For example, the attachment post 410 of the retractor blade 60 may be implemented as one or more tabs and the attachment port 312 of the retractor connector 50 may be implemented as one or more slots that are adapted to receive one or more tabs of the retractor blade 60. Furthermore, in some embodiments, the retractor connector 50 may include one or more openings (not shown) in sidewalls 308 of the retractor connector to permit a sideways loading the attachment post 410 into the attachment port 312 through one or such openings in the sidewalls 308.

As shown in FIG. 3B, the handle arm segment 48 may include a handle mount 516. The handle mount 516 may be sized and adapted to provide a location for attachment of the handle 80. A person may use the attached handle 80 to position the articulated arm 40 and a retractor blade 60 attached thereto.

Referring to FIG. 6, the handle mount 516 may comprise a generally, flat tab 518 having an upper side 511 and a lower side 513 that both extend parallel to an upper side 49 of the handle arm segment 48. A pedestal 519 may affix the lower side 513 of the tab 518 to the upper side 49 of the handle arm segment 48. In particular, the pedestal 519 may have a smaller width and length than the tab 518 such that lateral sides 521 and a distal end 523 of the tab 518 extend beyond the pedestal 519.

The tab 518 on the pedestal 519 may therefore define grooves 517 between the lower side 513 of the tab 518 and the upper side 49 of the handle arm segment 48. In particular, the grooves 517 may traverse along the lateral sides 521 of the tab 518 and may traverse parallel along the upper side 49 of the handle arm segment 48. As explained below, the handle 80 may be detachably coupled to the handle arm segment 48 via a slidable engagement of the handle 80 along the grooves 517. To this end, the distal end 523 of the tab 518 may be rounded or tapered to ease insertion of the tab 518 into an attachment portion 610 of the handle 80. Furthermore, the upper side 511 of the tab 518 may include a tab recess 525. The tab recess 525 is sized to receive a rod 660 of the handle 80. When received, the rod 660 may be advanced to a locked position in which the rod engage walls of the tab recess 525 and prevents removal of the tab 518 from the attachment portion 610 of the handle 80. Thus, when advanced to the locked position, the rod 660 may lock the handle 80 to the handle arm segment 48.

In the depicted embodiments, the lateral sides 521 of the tab 518 are aligned or parallel with a longitudinal axis of the handle arm segment 48. However, in some embodiments, the lateral sides 521 may be aligned perpendicular to or at some other angle with regard to the longitudinal axis of the handle arm segment 38. Moreover, the handle mount 516 is depicted attached to an upper surface of the handle arm segment 48 with upper and lower sides 511, 513 parallel with the upper surface of the handle arm segment 48. However, in some embodiment the handle mount 516 may be attached to other surfaces of the handle arm segment 48 and/or at an angle with regard to the attached surface. The angle and/or surface to which the handle mount 516 is attached may be adjusted to better accommodate forces applied to the attached handle 80 for various procedures. Furthermore, the tab 518 may have other shapes such as circular, oval, etc. which may accommodate attaching the handle 80 from multiple angles.

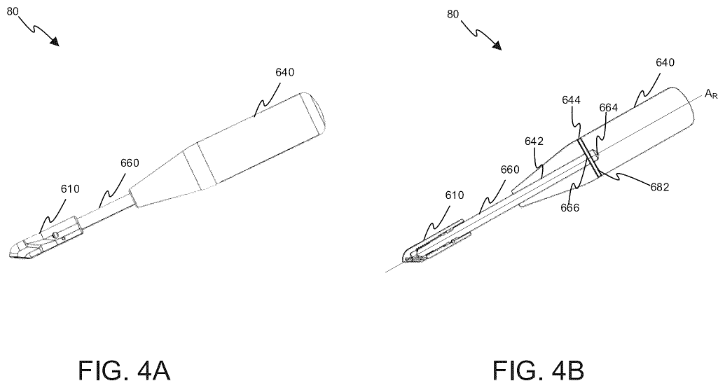

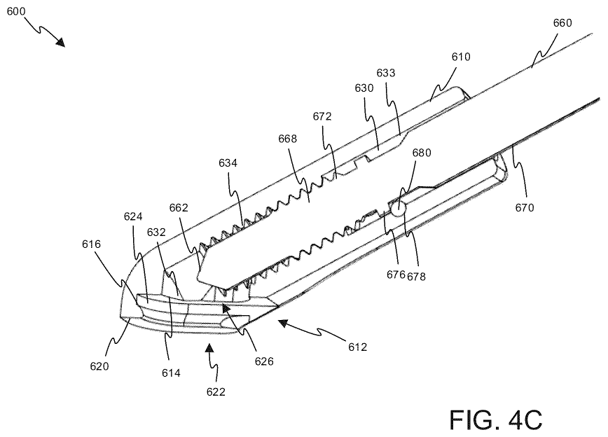

Referring not to FIGS. 4A-4C, the handle 80 may include an attachment portion 610, a hand grip 640, and a rod 660. The rod 660 may secure the hand grip 640 to the attachment portion 610. Moreover, the rod 660 may be manipulated to selectively engage the rod 660 with the walls of the tab recess 525.

The attachment portion 610 may be sized and adapted to cooperate with the handle mount 516 of the handle arm segment 48. The attachment portion 610 may comprise a slot 612 that is sized and adapted to accept and mate with the tab 518 and grooves 517 of the handle mount 516. The slot 612 and tab 518 may cooperate to secure the attachment portion 610 to the handle arm segment 48. To this end, the slot 612 may comprise an upper side 614 and a lower side 616. The lower side 616 may be spaced apart from the upper side 614 by a distance slightly larger than a thickness of the tab 518 of the handle mount 516. In this manner, the tab 518 of the handle mount 516 may be received by the slot 612 such that the sides 614, 616 of the attachment portion 610 closely mate and engage the sides 511, 513 of the handle mount 516.

Furthermore, the lower side 616 of the slot 612 may include an opening 622 sized to receive and closely mate with the pedestal 519 upon which the tab 518 rests. The slot 612 may comprise an end wall 624. The end wall 624 may be sized to receive and closely mate with the rounded or tapered distal end 523 of the tab 518. In particular, the end wall 624 and distal end 523 may cooperate to properly position the tab 518 within the slot 612. In particular, the end wall 624 may stop further advancement of the distal end 523 into the slot 612 when the tab recess 525 is properly aligned to receive the rod 660.

Finally, the upper side 614 of the slot 612 includes an aperture 626. In particular, the aperture 626 is positioned in the upper side 614 of the attachment portion 610 such that the aperture 626 aligns with the tab recess 525 when the tab 518 is fully inserted into the slot 612. The aperture 626 is sized to closely mate with a tip 662 of the rod 660. As shown, the tip 662 may be beveled or tapered. Such tapering may help guide the tip 662 into the tab recess 525 even in the presence of minor misalignment of the tab recess 525 with the aperture 626. For example, a person may fail to fully insert the tab 518 into the slot 612. The tapered tip 662 may aid the rod 660 in sliding into the tab recess 525 and urging the tab 518 into a fully inserted position.

As shown, the attachment portion 610 may include a longitudinal cavity 630 that is sized and adapted to receive a threaded end 668 of the rod 660. As shown, a distal end 632 of the longitudinal cavity 630 may adjoin the aperture 626 positioned in the upper side 614 of the attachment portion 610. The longitudinal cavity 630 may be shaped and sized such that its inner walls 633 closely mate with side walls 670 of the rod 660 and permit the rod 660 to slide longitudinally along at least a portion of the longitudinal cavity 630. The inner walls 633 may include threads 634 toward the distal end 632. The threads 634 are configured to engage threads 672 of the rod 660.

As a result of such threads 634, 672, the rod 660 may be advanced through the longitudinal cavity 630 and into the aperture 626 positioned in the upper side 614 of the attachment portion 610 via rotation of the rod 660 in a first direction about a longitudinal axis A_{R}. Conversely, rotation of the rod 660 in a second direction opposite the first direction may withdrawal the rod 660 from the aperture 626.

With reference to FIGS. 3A and 4C, as the rod 660 is advanced into the aperture 626, a tapered end 632 of the rod 660 may engage the tab recess 525 of tab 518 and prevent withdrawal of the tab 518 from the attachment portion 610. Once in the locked position, an annular rib 676 of rod 660 clears an aperture 678 in the attachment portion 610 that extends laterally into the longitudinal cavity 630. A pin 680 may then be inserted into the aperture 678. The pin 680 may engage and block the passage of the annular rib 676 and thereby prevent withdrawal of the rod 660 from the aperture 626. In this manner, the rod 660 may be secured to the handle mount 516 of the handle arm segment 48 and thus prevent the detachment of the handle 80 from the handle arm segment 48.

The hand grip 640 may be sized and adapted to be grasped by a person. The hand grip 640 may include an aperture 642 sized to receive a proximal end 664 of the rod 660. As shown in FIG. 4B, the hand grip 640 may include an aperture 644 which may be aligned with a corresponding aperture 666 toward the proximal end 664 of the rod 660. Another pin 682 may be passed through the apertures 644, 666 thereby locking the hand grip 640 to the rod 660. As such, a person may rotate the hand grip 640 about the longitudinal axis A_{R} of the rod 660 in order to advance the rod 660 through the aperture 626.

After the handle 80 is secured to the handle arm segment 48, the hand grip 640 provides for convenient manipulation and placement of the articulated arm 40 and an attached retractor blade 60. After the articulated arm 40 and retractor blade 60 are positioned as desired, a person may remove the pin 680 from the attachment portion 610. The person may then rotate the hand grip 640 about longitudinal axis A_{R} to withdrawal the rod 660 from the aperture 626 and disengage the rod 660 from the handle mount 516. After such disengagement, the handle 80 may be detached from the handle arm segment 48 by simply sliding the attachment portion 610 off the tab 518 of the handle mount 516.

The following provides an exemplary process for using the retractor system 10. The process may provide insight regarding various aspects of the retractor system 10. However, the order of several of the steps is not rigid and various steps may be performed in a different order without deviating from the spirit and scope of the pending claims. The process may begin with securing the mounting assemblies 20 to the hospital bed before or after a patient is placed on the hospital bed. Similarly, articulated arms 40 may be secured to the mounting assemblies 20 before or after the mounting assemblies are secured to the hospital bed. A person may select a suitable retractor blade 60 and secure the retractor blade 60 to the blade connector 50 of an articulated arm 40. Furthermore, the attachment portion 610 of a handle 80 may be slid over a tab 518 of a handle mount 516 and then hand grip 640 may be rotated to advance the rod 660 and lock the handle 80 to the handle mount 516. The retractor blade 60 and articulated arm 40 may be manipulated with the aid of the attached handle 80 so as to insert a distal end of the retractor blade 60 into the operative site and position the retractor blade 60 and/or articulated arm 50 as desired to retract tissue and provide access to the surgical site of interest. After positioned as desired, the hand grip 640 may be rotated in the opposite direction so as to withdrawal the rod 660 and release the handle 80 from the handle mount 516. The attachment portion 610 of the released handle 80 may be slid off the tab 518 and removed from the handle mount 516. After removing the handle 80, the retractor blade 60 may be secured at both its distal end via the patient's anatomy and at proximal end via the articulated arm 40, thus removing the need for manual holding of the retractor blade 60 during the procedure.

While the articulated arm 40 generally maintains the retractor blade 60 in position, the motion permitted by the joints of the articulated arm 40 and/or the interaction between the retractor connector 50 and the upper annular groove 412 (if the upper annular groove 412 is utilized) allows some amount of “float” for the retractor blade 60 relative to the hospital bed in the event of any pounding, chiseling, or other events that may cause portions of the anatomy or equipment to shift, helping to maintain a desired access shape as well as helping to reduce risk of any additional injury or trauma to the patient, as well as damage to any equipment, that may be caused by such a shift or movement. Additional retractor blades 60 and articulated arm 40 may be added, positioned, and secured in place in a similar manner. Thus, the retractor system 10 provides for flexibility in the formation of the desired access site, as well as, reducing obstacles to accessing the site of interest.

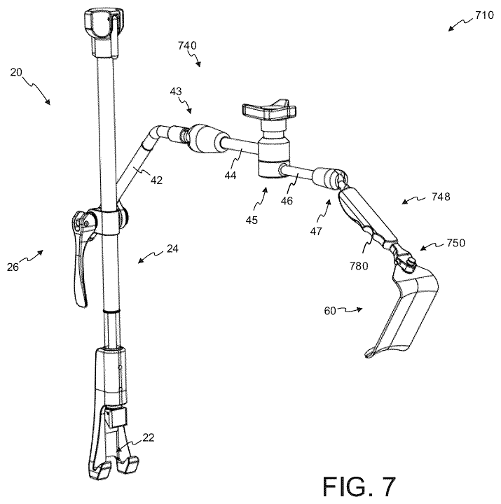

FIG. 7 illustrates another embodiment of a retractor system 710 in accordance with various aspects of the present disclosure. The retractor system 710 may include a mounting assembly 20, an articulated arm 740 with a handle segment 748, and a retractor blade 60. The mounting assembly 20 and retractor blade 60 may be implemented in the same manner as the mounting assembly 20 and retractor blade 60 of the retractor system 10 shown in FIG. 1. FIG. 7 depicts a single mounting assembly 20 with a single articulated arm 740 attached to the mounting assembly 20. However, in some embodiments, the retractor system 710 may include a different number of mounting assemblies 20, a different number of articulated arms 40 attached to each mounting assembly 20, and/or a different number of articulated arms 740 attached to each mounting assembly 20.

The articulated arm 740 may adjustably constrain the retractor blade 60 secured to the distal end of the articulated arm 740. In some embodiments, the articulated arm 740 may provide the retractor blade 60 with at least some freedom of movement relative to the post 24 and the bed mount 22. In particular, the articulated arm 740 may allow the retractor blade 60 some limited movement but may generally maintain the retractor blade 60 at or near a selected position. In other embodiments, the articulated arm 740 may be adapted to maintain the retractor blade 60 in a substantially fixed or stationary position once the articulated arm 740 is positioned and locked into place.

To this end, the articulated arm 740 may comprise multiple arm segments and joints that permit articulating the arm segments. In particular, the multiple arm segments may be sequentially adjoined such that a proximal end of one segment is coupled to a distal end of a proceeding segment. In this manner, the multiple arm segments may span from a proximal end the articulated arm 740 to a retractor connector 750 at a distal end of the articulated arm 740. More specifically, the articulated arm 740 may include a shoulder arm segment 42. A distal end of the shoulder arm segment 42 may be joined to a proximal end of an upper arm segment 44 by a shoulder joint 43. Moreover, a distal end of the upper arm segment 44 may be joined to a proximal end of a fore arm segment 46 by an elbow joint 45. Finally, a distal end of the fore arm segment 46 may be joined to a proximal end of a handle segment 748 by a wrist joint 47.

The arm segments 42, 44, and 46 are generally referred to herein as a “shoulder” arm segment, an “upper” arm segment, a “fore” arm segment in a manner similar to a human arm. Similarly, joints 43, 45, 47 are generally referred to herein as a “shoulder” joint, an “elbow” joint, and a “wrist” joint in a manner similar to joints of a human arm. However, such terms are merely used as a manner of convenience. This analogy to a human arm is especially helpful with regard to the depicted embodiment in which the joints 43, 47 are ball joints and the joint 45 is a pivot joint. Such joints provide the articulated arm 740 with articulation similar to the human arm. However, the articulated arm 740, in some embodiments, may include a fewer number of arm segments or a greater number of arm segments. Similarly, the joints of the articulated arm 740 may not align with joints of the human arm and may comprise some combination of single-axis hinge joints, pivot joints, universal joints, ball joints, prismatic joints, and/or other types of joints.

Each arm segment 44, 46, 748 of articulated arm 740 may comprise one or more generally straight, elongated members. Similarly, the shoulder arm segment 42 may include one or more elongated members. The elongated members of the shoulder arm segment 42 may include one or more bends or curves and/or may be coupled to one another to provide the shoulder arm segment 42 with one or more bends or curves. In some embodiments, each arm segment 42, 44, 46, 748 may comprise a selection of straight and/or bent members so as to accommodate different procedures or access sites. In various embodiments, the elongated members of the arm segments 44, 46, 748 may have a cylindrical shape. Such cylindrical members may have circular cross sections, a non-circular cross sections, and/or irregular cross sections.

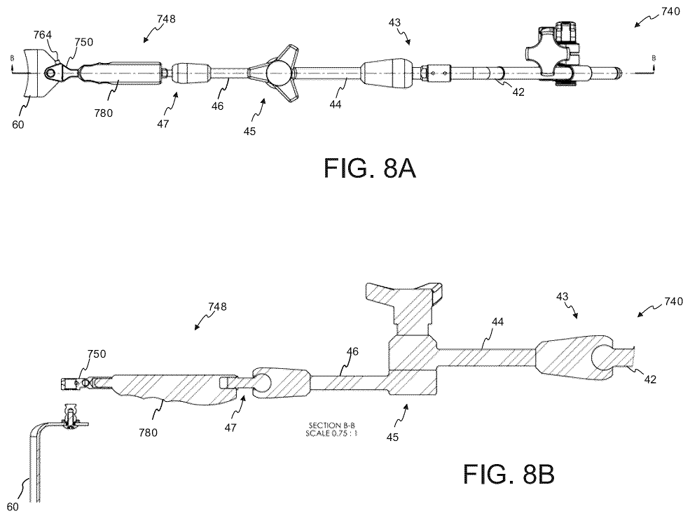

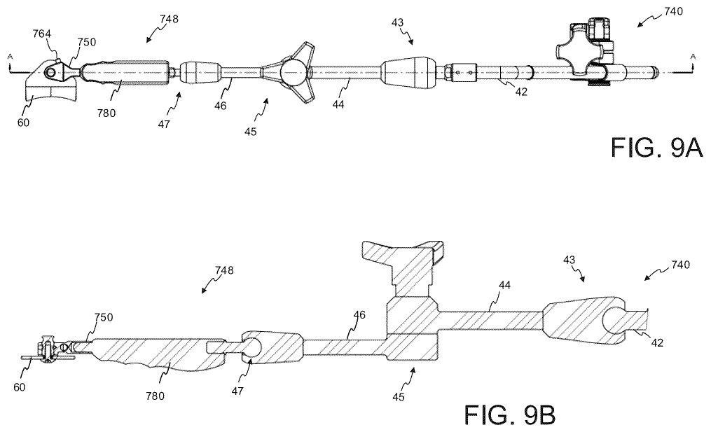

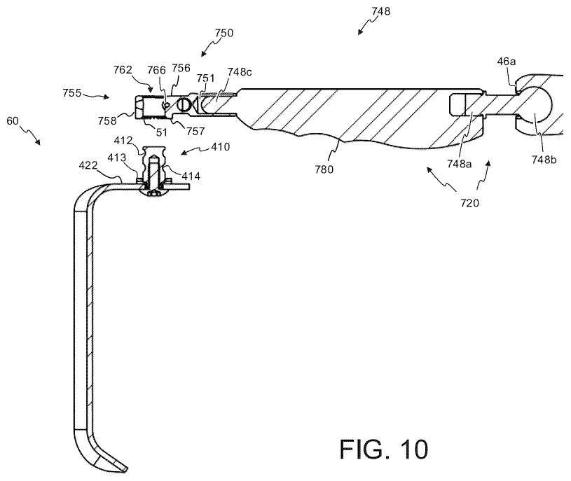

As shown in FIGS. 7-10, the fore arm segment 46 and handle segment 748 may be joined by the wrist joint 47. In particular, a distal end of the fore arm segment 46 may include a socket 46a of the wrist joint 47. Moreover, a proximal end of the handle segment 748 may include a shaft 748a, which is coupled to a ball 748b of the wrist joint 47. However, in other embodiments, the proximal end of the handle segment 748 may include the socket of the wrist joint 47 and the distal end of the fore arm segment 46 may include a shaft attached to a ball of the wrist joint 47.

The ball 748b may engage internal sides of the socket 46a and the socket 46a may prevent the ball 748b from being withdrawn from socket 46a. However, the socket 46a may otherwise permit the ball 748b to rotate within the socket 46a and along the internal sides of the socket 46a. As such, the wrist joint 47 may permit articulation of the handle segment 748 with respect to the fore arm segment 46 about multiple axes or an indefinite number axes. In some embodiments, the wrist joint 47 may constrain or otherwise limit a degree of movement of the handle segment 748 about one or more axes. For example, as shown in FIG. 7, the wrist joint 47 may be oriented such that an opening 46b of the socket 46a is narrower in the horizontal direction than in the verticle direction. In such an embodiment, left and right sides of the socket opening 46b may engage the shaft 748a and limit articulation of the handle segment 748 in a leftward or rightward direction. Conversely, upper and lower sides of the socket opening 46b may engage the shaft 748a and limit articulation of the handle segment 748 in an upward or downward direction, but permit greater articulation in such directions than what is permitted in the leftward or rightward directions.

As noted above, the handle segment 748 may include one or more elongated members. In particular, the handle segment 748 may include elongated members 720 that are sequentially coupled to one another such that the elongated members 720 extend from a proximal end of the handle segment 748 to a distal end of the handle segment 748. Moreover, a distal end of the sequentially coupled elongated members 720 may be coupled to a retractor connector 750. In particular, a proximal end of the retractor connector 750 may include a cylindrical port 751. The cylindrical port 751 may be sized to receive and affix a cylindrical post 748c at the distal end of one of the elongated members 720 to the proximal end of the retractor connector 750.

A distal end of the retractor connector 750 may include a connector head 755. The connector head 755 may include an upper side 756, a lower side 757, and sidewalls 758 that couple the upper side 756 to the lower side 757. The connector head 305 may include an attachment port 762 through the upper side 756 and the lower side 757, a release button 764, and a locking ball 766. In the illustrated embodiment, the attachment port 762, release button 764, and locking ball 766 cooperate to detachably couple and lock the retractor blade 60 to the articulated arm 740 in a manner similar to the connector head 305.

Through the use of an internal mechanism (not shown), depressing the release button 764 allows increased movement of the locking ball 766, which permits an attachment post 410 of retractor blade 60 to be inserted into or removed from the attachment port 762. Releasing the release button 764 causes the release button 764 and internal mechanism to return to a locked position in which the internal mechanism forces the locking ball 766 into the attachment port 762 such that the locking ball 766 engages the attachment post 410 of the retractor blade 60 and secures the retractor blade 60 to the retractor connector 750. Thus, a retractor blade 60 may be secured to the articulated arm 40 and a hospital bed via a retractor connector 750.

In the depicted embodiments, the attachment port 762 of the retractor connector 750 and the attachment post 410 of the retractor blade 60 are cylindrical with circular cross-sections. Such an embodiment may permit swiveling of the retractor blade 60 with regard to the retractor connector 750, even after attachment. However, such swiveling of the retractor blade 60 may not be required or may not be desired for certain surgical procedures. As such, the attachment port 762 and the attachment post 410 may be shaped in a manner that prevents such swiveling. For example, the attachment port 762 and the attachment post 410 may remain cylindrical but have a non-circular cross-section (e.g., square, rectangular, hexagonal, etc.). In yet other embodiments, the attachment port 762 and attachment post 410 may not by cylindrical, but may otherwise provide a mating engagement of the retractor blade 60 to the retractor connector 750. For example, the attachment post 410 of the retractor blade 60 may be implemented as one or more tabs and the attachment port 762 of the retractor connector 750 may be implemented as one or more slots that are adapted to receive one or more tabs of the retractor blade 60. Furthermore, in some embodiments, the retractor connector 750 may include one or more openings (not shown) in sidewalls 758 of the retractor connector 750 to permit a sideways loading the attachment post 410 into the attachment port 762 through one or such openings in the sidewalls 758.

As noted above, the handle segment 748 may comprise one or more elongated members 720. In particular, the handle segment 748 as shown in FIGS. 7-10 may comprise one or more elongated members 720 that define a generally straight, cylindrical member or arm segment that extends from a proximal end of the handle segment 748 to a distal end of the handle segment 748. However, in some embodiments, one or more members 720 of the handle segment 748 may include one or more bends or curves so as to define an arm segment with one or more bends or curves between its proximal and distal ends.

As shown, the handle segment 748 may include a handle 780 between its distal and proximal ends. In particular, the handle 780 may closely conform with an outer surface of the one or more elongated members 720 so as to provide a handle 780 having a distal end and a proximal end that are in-line with distal and proximal ends of the handle segment 748. To this end, the handle 780 may be molded over one or more of the elongated members 720 via an over-mold process. Such process may encapsulate or surround outer surfaces of the elongated members 720 in a molded material (e.g., plastic, resin, rubber, metal, etc.). Thus, the resulting handle 780 may have an inner surface that conforms to and/or circumscribes outer surfaces of one or more elongated members 720. In this manner, a longitudinal axis of the handle 780 may parallel or coaxially align with a longitudinal axis of the handle segment 748 and/or a longitudinal axis of one or more of the elongated members 720.

In other embodiments, such as the embodiment shown in FIGS. 7-10, the handle 780 may be integral with one or more elongated members 720 of the handle segment 748. In such embodiments, a longitudinal axis of the handle 780 may still parallel or coaxially align with a longitudinal axis of the handle segment 748 and/or one or more of the elongated members 720.

As explained herein, a person may use the handle 780 to position the articulated arm 740 and/or retractor blade 60 attached thereto. As such, the handle 780 may be sized and adapted to be grasped by a person. To this end, the handle 780 may include one or more depressions that align with a person's fingers when grasped. Moreover, a surface of the handle 780 may textured to better engage a person's hands, palms, and/or fingers when grasped. Such texturing may reduce slippage and/or otherwise aid the person in maintaining a firm grip of the handle 780 when positioning the articulated arm 740 and/or retractor blade 60. In some embodiments, the articulated arm 740 may generally comprise elongated members having a generally uniform circumference and/or a generally small circumference. In such embodiments, the handle 780 may provide the articulated arm 740 with a greater circumference that longitudinally traverse a portion of the articulated arm 740. Such greater circumference may enable a person to more easily grasp and apply force to the articulated arm 740 than the smaller circumferences provided by the elongated members. Thus, the handle 780 may provide a convenient location for a person to grasp the articulated arm 740 and manipulate the articulated arm 740 and/or an attached retractor blade 60 to a desired position.

The following provides an exemplary process for using the retractor system 710. The process may provide insight regarding various aspects of the retractor system 710. However, the order of several of the steps is not rigid and various steps may be performed in a different order without deviating from the spirit and scope of the pending claims. The process may begin with securing the mounting assemblies 20 to the hospital bed before or after a patient is placed on the hospital bed. Similarly, articulated arms 740 may be secured to the mounting assemblies 20 before or after the mounting assemblies are secured to the hospital bed. A person may select a suitable retractor blade 60 and secure the retractor blade 60 to the blade connector 750 of an articulated arm 740. The retractor blade 60 and articulated arm 40 may be manipulated with the aid of the handle 780 so as to insert a distal end of the retractor blade 60 into the operative site and position the retractor blade 60 and/or articulated arm 50 as desired to retract tissue and provide access to the surgical site of interest. As a result of such positioning, the retractor blade 60 may be secured at both its distal end via the patient's anatomy and at proximal end via the articulated arm 740, thus removing the need for manual holding of the retractor blade 60 during the procedure.

Unlike the retractor system 10 described above, a person does not attach a handle 780 to the articulated arm 740 in order to manipulate the articulated arm 740 and/or attached retractor blade 60. Moreover, a person does not need to detach the handle 780 after positioning the articulated arm 740 and/or attached retractor blade 60. As explained above, the handle 780 is in-line with the distal end and proximal end of the handle segment 748 and generally conforms to its elongated members 720. In particular, the handle 780 does not appreciably add to the profile of the handle segment 748 and its elongated members 720. As such, the handle 780 does not obstruct or appreciably obstruct visual and/or physical access to the surgical site and does not need to be removed.

While the articulated arm 740 generally maintains the retractor blade 60 in position, the motion permitted by the joints of the articulated arm 740 and/or the interaction between the retractor connector 750 and the upper annular groove 412 (if the upper annular groove 412 is utilized) allows some amount of “float” for the retractor blade 60 relative to the hospital bed in the event of any pounding, chiseling, or other events that may cause portions of the anatomy or equipment to shift, helping to maintain a desired access shape as well as helping to reduce risk of any additional injury or trauma to the patient, as well as damage to any equipment, that may be caused by such a shift or movement. Additional retractor blades 60 and articulated arms 740 may be added, positioned, and secured in place in a similar manner. Thus, the retractor system 10 may provide flexibility in the formation of the desired access site, as well as, reducing obstacles to accessing the site of interest.

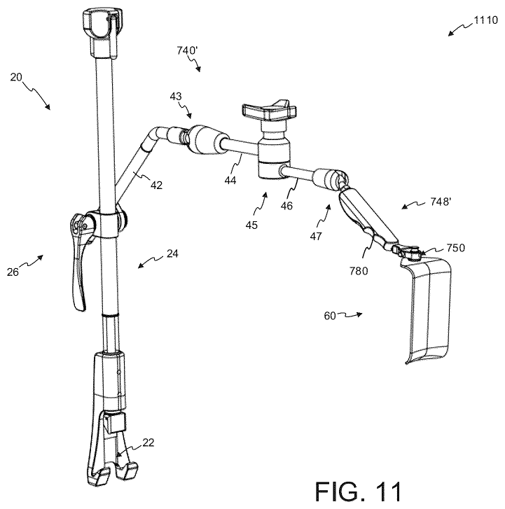

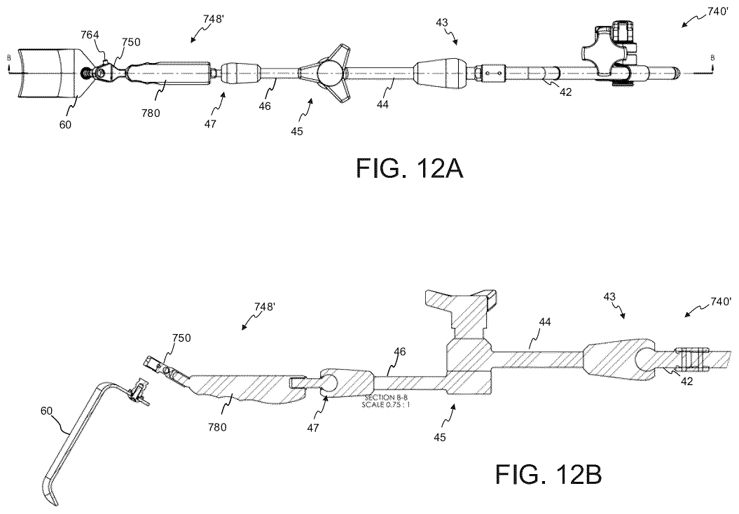

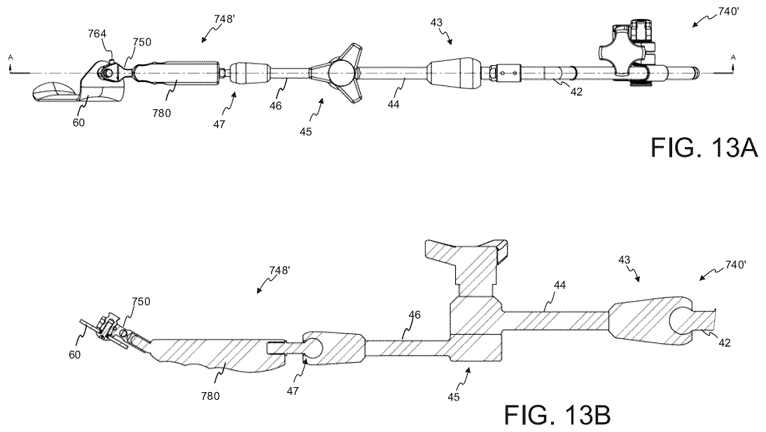

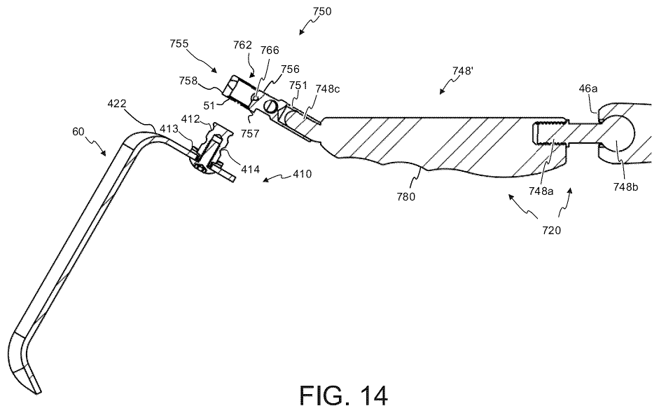

FIGS. 11-14 illustrate another embodiment of a retractor system 1110 in accordance with various aspects of the present disclosure. The retractor system 1110 may be implemented in a similar manner as the retractor systems 710 of FIGS. 7-10. Like the retractor system 710, the retractor system 1110 may include a mounting assembly 20, an articulated arm 740′ with a handle segment 748′, and a retractor blade 60. However, the articulated arm 740′ and its handle segment 748′ differ from the articulated arm 740 and its handle segment 748 in that the handle segment 748′ is angled with respect to the retractor connector 750. In particular, the handle segment 748′ is angled such that the handle segment 748′ extends from the retractor connector 750 in an upward direction.

Such angling may provide additional space between the handle 780 of the handle segment 748′ and a patient. When a distal end of the retractor blade 60 is inserted into an operative site and positioned, the proximal end of the retractor blade 60 and the retractor connector 750 may rest upon the skin of the patient. With such a placement, an in-line handle segment 748 such as shown in FIG. 7 may rest its handle 780 upon the skin of the patient or may place its handle 780 in close proximity to the skin of the patient. However, a handle segment 748 that angles in an upward direction as it extends from the retractor connector 750 may position its handle 780 away from the skin of the patient. Due to such additional clearance between the handle 780 and the patient, a person may find it easier to properly position the retractor blade 60 using the angled handle segment 748′ of FIGS. 11-14 than using the in-line handle segment 748 of FIGS. 7-10.

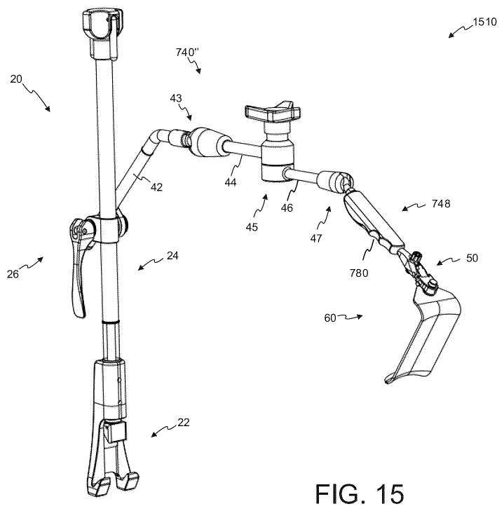

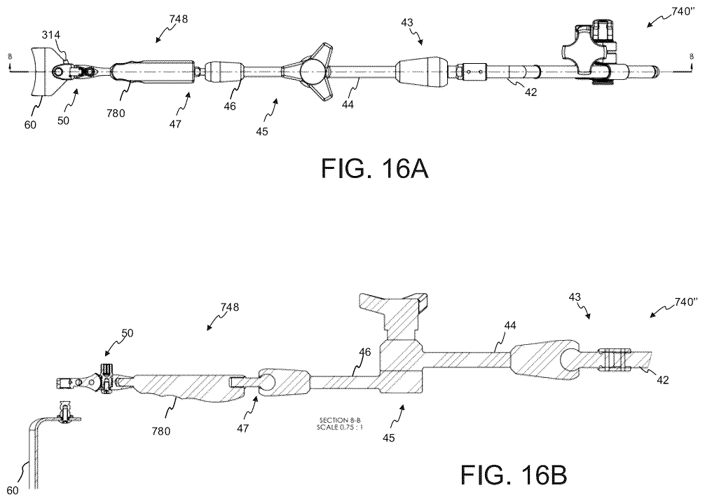

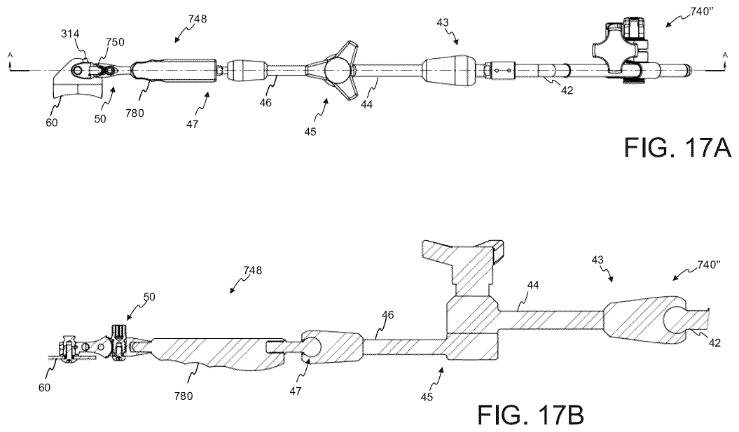

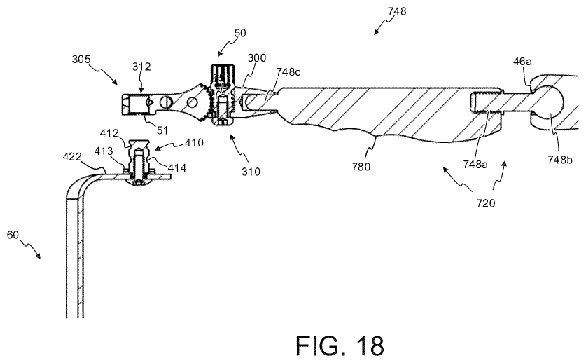

FIGS. 15-18 illustrate another embodiment of a retractor system 1510 in accordance with various aspects of the present disclosure. The retractor system 1510 may be implemented in a similar manner as the retractor systems 710 of FIGS. 7-10. Like the retractor system 710, the retractor system 1510 may include a mounting assembly 20, an articulated arm 740″ with a handle segment 748, and a retractor blade 60. However, the articulated arm 740″ may differ from the articulated arm 740 in that the handle segment 748′ is coupled to a retractor connector 50 having an adjustable pivot 310. As described with regard to the retractor system 10 of FIG. 1, the adjustable pivot 310 of the retractor connector 50 may permit adjusting an angle between a connector body 300 and the connector head 305 of the retractor connector 50. Thus, despite the handle segment 748 of articulated arm 740″ comprising an in-line handle segment like the articulated arm 740 of FIG. 7, the handle segment 748 may be selectively angled in an upward direction with respective to the connector head 305 via the adjustable pivot 310. Such angling may provide additional space between the handle 780 of the articulated arm 740″ and a patient in a manner similar to the retractor system 1110. Moreover, the adjustable pivot 310 may also aid adjusting the angle of the attached retractor blade 60 in a manner similar to the retractor system 10 of FIG. 1.

While particular embodiments of the invention have been shown, it will be understood that the invention is not limited thereto since modifications may be made by those skilled in the art, particularly in light of the foregoing teaching. It is therefore, the appended claims that define the true spirit and scope of the invention.

What is claimed is:

1. An articulated arm, comprising:
a first arm segment comprising a first arm segment proximal end and a first arm segment distal end;
a retractor connector configured to engage and retain a retractor blade; and
a handle segment comprising a handle and one or more handle segment members;
wherein a proximal end of the one or more handle segment members is coupled to the first arm segment distal end;
wherein a distal end of the one or more handle segment members is coupled to the retractor connector; and
wherein the handle extends longitudinally along at least one of the one or more handle segment members.

2. The articulated arm of claim 1, wherein the handle comprises material molded over the at least one of the handle segment members.

3. The articulated arm of claim 1, wherein the handle is integral with the at least one of the handle segment members.

4. The articulated arm of claim 1, wherein each handle segment member comprises an elongated member.

5. The articulated arm of claim 1, wherein:
the at least one of the handle segment members has a first circumference; and
the handle has a second circumference that is greater than the first circumference.

6. The articulated arm of claim 1, wherein the handle comprises depressions.

7. The articulated arm of claim 1, wherein a longitudinal axis of the handle is parallel to a longitudinal axis of the at least one of the handle segment members.

8. The articulated arm of claim 1, wherein a longitudinal axis of the handle is co-axial with a longitudinal axis of the at least one of the handle segment members.

9. The articulated arm of claim 1, wherein the retractor connector comprises an attachment port configured to receive an attachment post of the retractor blade.

10. The articulated arm of claim 9, wherein:
the retractor connector engages an annular groove of the attachment post when the attachment post is received by the attachment port; and
wherein engagement of the annular groove by the retractor connector prevents withdraw of the attachment post from the attachment port.

11. The articulated arm of claim 1, comprising a joint that permits articulation of the handle segment with respect to the first arm segment.

12. The articulated arm of claim 11, wherein the joint comprises a ball attached to one of the first arm segment and the handle segment and a socket attached to the other of the first arm segment and the handle segment.

13. The articulated arm of claim 11, wherein an angle between the handle and the retractor connector positions a lower side of the handle higher than a lower side of the retractor connector.

14. A retractor system, comprising:
a mounting assembly comprising a post and a bed mount;
an articulated arm comprising a plurality of arm segments, a retractor connector, and a handle segment; and
a retractor blade attached to the retractor connector;
wherein a proximal end of the plurality of arm segments is coupled to the post;
wherein a proximal end of the handle segment is coupled to a distal end of the plurality of arm segments;
wherein a distal end of the handle segment is coupled to the retractor connector; and
wherein the handle segment comprises a handle that extends longitudinally along the handle segment.

15. The retractor system of claim 14, wherein:
the handle segment comprises an elongated member between the proximal end and the distal end of the handle segment; and
the handle comprises material molded over the elongated member.

16. The retractor system of claim 14, wherein:
the handle segment comprises an elongated member between the proximal end and the distal end of the handle segment; and
the handle is integral with the elongated member.

17. The retractor system of claim 14, wherein:
the handle segment comprises an elongated member between the proximal end and the distal end of the handle segment;
the elongated member has a first circumference; and
the handle has a second circumference that is greater than the first circumference.

18. The retractor system of claim 14, wherein a longitudinal axis of the handle is parallel to a longitudinal axis of the handle segment.

19. The retractor system of claim 14, wherein a longitudinal axis of the handle is co-axial with a longitudinal axis of the handle segment.

20. The retractor system of claim 14, wherein:
the retractor blade comprises an attachment post;
the retractor connector comprises an attachment port configured to receive the attachment post of the retractor blade;
the retractor connector permits rotation of the retractor blade about a longitudinal axis of the attachment post when coupled to the retractor connector.
